(19) United States
(12) Patent Application Publication
(10) Pub. No.: US 2013/0096166 A1
(43) Pub. Date: Apr. 18, 2013

(54) CAMALEXIN AS A TREATMENT FOR PROSTATE CANCER

(71) Applicant: Clark Atlanta University, Atlanta, GA (US)

(72) Inventors: Valerie Odero-Marah, Tucker, GA (US); Basil Smith, Northport, FL (US)

(73) Assignee: Clark Atlanta University, Atlanta, GA (US)

(21) Appl. No.: 13/653,802

(22) Filed: Oct. 17, 2012

Related U.S. Application Data
(60) Provisional application No. 61/547,755, filed on Oct. 17, 2011.

(51) Int. Cl. A61K 31/427; C12N 5/09; A61P 35/00

(52) U.S. Cl. 514/365; 435/375

(57) ABSTRACT

Compositions and methods to treat prostate cancer with the compound camalexin and its structural analogs. Camalexin increases ROS production and decreases the proliferation of prostate cancer cells, especially aggressive prostate cancer cells.

### STATEMENT REGARDING FEDERALLY SPONSORED RESEARCH OR DEVELOPMENT

This invention was made with government support under NIH grants 1P20MD002285 and G12RR03062. The government has certain rights in the invention.

### BACKGROUND OF THE INVENTION

Prostate cancer (PCa) is a leading cause of non-cutaneous malignancy in men in Western industrialized nations and originates almost always from the glandular epithelia of the organ. Although prostate cancer mortality has been declining in recent years, it is still a major cause of cancer death in men in the United States. With current treatment options, including surgical or medical castration (leuprolide and flutamide), the 5-year survival rate for men diagnosed with organ-confined PCa is approaching 100%; however, the prognosis becomes worse once the cancer extends beyond the prostatic capsule. Once the cancer metastasizes to other organs, a majority of patients die from their tumors as opposed to other causes, which is due in part to the tumor ultimately becoming androgen-independent within a median of 18 to 24 months after castration. Taxanes and estramustine have been increasingly used for cytotoxic chemotherapy in these patients, but unfortunately, their therapeutic efficacy is limited and most hormone refractory prostate cancer patients treated with these chemotherapeutic agents manifest resistance and eventually succumb to the disease. Therefore, metastatic PCa remains the main cause of prostate cancer related death in men.

Numerous epidemiological studies have shown an association between reduced cancer risk and intake of a diet rich in phytochemicals from fruits and vegetable. Camalexin is a natural phytoalexin and the principal one found in Arabidopsis thaliana, Camelina sativa, and Capsella bursapastoralis, where it accumulates in response to environmental stress and reportedly displays antimicrobial activities against various plant pathogens. This compound has been researched predominantly with regard to plant chemical defense mechanisms, and has been shown to exert cytostatic/cytotoxic effects on the human protozoan pathogen Trypanosoma cruzi, and on the human breast cancer cell line SKBr3 where it displays higher potency than the traditional anticancer agents cisplatin and melphalan. Mezencev et al. showed that camalexin induced apoptosis in Jurkat T-leukemia cells by increasing concentration of ROS and activation of caspases. However, its cytotoxicity against eukaryotic cells and potential as a human disease modifying drug has been examined in a limited context and its role in prostate cancer has never been investigated.

Current interest in epithelial to mesenchymal transition (EMT) stems from its developmental importance and its involvement in several adult pathologies including prostate cancer where it provides a mechanism for tumor cells to become more motile, invasive, and metastatic. This involves loss of epithelial characteristics such as decreased expression of E-cadherin and cytokeratins, and acquisition of mesenchymal markers such as increased expression of vimentin and N-cadherin, as the cells become more invasive and metastatic. Snail transcription factor, a member of the Snail superfamily, is a zinc finger protein that can downregulate E-cadherin by binding several E-boxes located in the promoter region, thus promoting EMT. Human cancer development is associated with chronic inflammation with the release of reactive oxygen species (ROS) by inflammatory cells resulting in DNA damage. ROS has been associated with EMT and it has been shown that TGF-β induces EMT via up-regulation of ROS species, hydrogen peroxide (H₂O₂), and MAPK ERK signaling in proximal tubular epithelial cells. A human prostate cancer EMT cell model has been established by overexpressing Snail transcription factor in ARCaP cells, and shows that Snail-mediated EMT is partly regulated by increased ROS and ERK signaling and leads to increased tumorigenicity. Hydrogen peroxide has been shown to increase progressively in the lesser aggressive LNCaP cells to its more aggressive sublines C4, C4-2 and C4-2B, with concomitant increase in tumorigenic and metastatic potential. Since increased levels of ROS in cancer cells is seen as inducing survival, many therapies in development are focused on reducing ROS levels. However, since ROS levels are already high in cancer cells, increasing the levels further may tip the balance and trigger cell death instead, by producing so much damage that the cell undergoes apoptosis or even necrosis.

### SUMMARY OF THE INVENTION

The present invention involves a compound and method for treating prostate cancer. The compound camalexin and its structural analogs has been shown to be effective in decreasing proliferation of prostate cancer cells.

In particular, more aggressive prostate cancer cells that express high levels of ROS are more sensitive to camalexin as compared to lesser aggressive prostate cancer cells. The method thus involves targeting more aggressive prostate cancer cells and increasing their production of ROS even higher to cause cell death.

Increased ROS in cancer cells has been associated with induction of epithelial to mesenchymal transition (EMT), a process that produces aggressiveness in tumor cells. More aggressive prostate cancer cells produce more ROS than the less aggressive cells. Hence, we hypothesized that camalexin may be a good treatment option for aggressive cancer cells that normally do not respond to treatment with anti-androgens or chemotherapy. Further induction of ROS production by camalexin treatment in aggressive prostate cancer cells that already express high levels of ROS may sensitize them to increased cytotoxicity as compared to less aggressive cancer cells.

Cell viability assays with camalexin were performed on several prostate cancer cell lines, including one in which Snail, a transcription factor, is overexpressed. Snail is responsible for the induction of EMT and increased production of ROS and thus contributes to a more aggressive potential of this cell line. Five cell lines were used: human prostate cancer cell line ARCaP stably transfected with constitutively active Snail cDNA (ARCaP Snail) representing an aggressive cell line; human prostate cancer cell line ARCaP stably transfected with empty vector Neo (ARCaP Neo) representing the lesser aggressive cell line; LNCaP cells with lesser metastatic potential; C4-2 cells which are a more aggressive subline of LNCaP cells; and normal prostate epithelial cells (PrEC).

We assayed these cell lines for cell proliferation in the presence of camalexin. We found that camalexin significantly decreased proliferation of the more aggressive C4-2 cells as compared to less aggressive LNCaP cells. Also, ARCaP Neo cells were less affected, while significant decrease in viability was noted for ARCaP Snail cells, which is the more aggressive counterpart. Camalexin did not decrease the viability of normal prostate epithelial cells.

We also assayed cells to determine degree of apoptotic cell death in the presence of camalexin. We found higher protein expression of cleaved PARP (the enzyme poly ADP ribose polymerase) in aggressive C4-2 cells treated with camalexin as compared to less aggressive LNCaP cells. This result indicates that increased cytotoxicity observed in aggressive C4-2 cells treated with camalexin can be attributed to induction of apoptosis subsequent to camalexin treatments.

### DESCRIPTION OF THE DRAWINGS

FIG. 1A is a bar chart comparing migration of ARCaP Snail cells, ARCaP Neo cells, C4-2 cells, and LNCaP cells.

FIG. 1B is a bar chart illustrating baseline ROS in ARCaP Snail cells, ARCaP Neo cells, C4-2 cells, and LNCaP cells.

FIG. 1C illustrates ROS production in C4-2 cells when treated with camalexin and upon addition of the ROS scavenger NAC and the positive control H₂O₂.

FIG. 1D illustrates ROS production in ARCaP Snail cells when treated with camalexin and upon addition of the ROS scavenger NAC and the positive control H₂O₂.

FIG. 2A illustrates cell viability of ARCaP Neo and ARCaP Snail cells at day 0 upon treatment with camalexin. FIG. 2B illustrates the cells at day 3. FIG. 2C illustrates cell viability of LNCaP and C4-2 cells at day 0 upon treatment with camalexin. FIG. 2D illustrates the cells at day 3. FIG. 2E illustrates cell viability of PrEC cells upon treatment with camalexin at days 0 and 3.

FIG. 3 illustrates caspase-3/7 activity in the ARCaP Snail cell line untreated and treated with 25 μM camalexin and 25 μM camalexin plus 10 mM NAC for 3 days.

FIG. 4A illustrates a western blot of ARCaP Neo and ARCaP Snail cells showing expression of the proteins Snail and caspase-3. β-actin was used as loading control. FIG. 4B shows quantification of protein amounts using densitometry with normalization to β-actin levels using the Quantity One quantification software (BioRad).

FIGS. 4C and 4D are similar figures for the cell lines LNCaP and C4-2.

FIG. 5A is a western blot of ARCaP Neo and ARCaP Snail cells showing cleaved PARP as a result of exposure to camalexin. FIG. 5B shows quantification of protein amounts using densitometry with normalization to β-actin levels using the Quantity One quantification software (BioRad). FIG. 5C is a western blot for LNCaP and C4-2 cells.

FIG. 6A shows bar charts of cell viability for ARCaP Snail cells at day 0 and day 3 of treatment with camalexin and NAC. FIG. 6B is a western blot of ARCaP Snail cells showing cleaved PARP as a result of exposure to camalexin and NAC. FIG. 6C shows bar charts of cell viability for ARCaP Neo cells at day 0 and day 3 of treatment with camalexin and H₂O₂. FIG. 6D is a western blot and quantification of ARCaP Neo cells showing cleaved PARP as a result of exposure to camalexin and H₂O₂.

FIG. 7A is bar charts showing cell viability for C4-2 cells at day 0 and day 3 of treatment with camalexin and NAC. FIG. 7B shows bar charts of cell viability for LNCaP cells at day 0 and day 3 of treatment with camalexin and H₂O₂. FIG. 7C is a western blot and quantification of LNCaP Neo cells showing cleaved PARP as a result of exposure to camalexin and H₂O₂.

### DETAILED DESCRIPTION

“Camalexin” as used herein refers to the phytoalexin 3-thiazol-2′yl-indole and structural analogs thereof. The term also refers to pharmaceutically acceptable salts of camalexin.

The term “effective amount” means that amount of a drug or pharmaceutical agent that will elicit the biological or medical response of a tissue, cell, system, animal, or human that is being sought by a researcher or clinician. The term “therapeutically effective amount” means any amount which, as compared to a corresponding subject who has not received such amount, results in improved treatment, healing, prevention, or amelioration of a disease or disorder, or a decrease in the rate of advancement of a disease or disorder, and also includes amounts effective to enhance normal physiological function.

The terms “treat” or “treatment” as applied to cancer refer to partially or totally inhibiting, delaying or preventing the progression of cancer including cancer metastasis; inhibiting, delaying, or preventing the recurrence of the cancer including cancer metastasis; or preventing the onset or development of cancer (chemoprevention) in a mammal, including humans.

The terms “patient” and “subject” as used herein refers to any vertebrate animal, preferably a mammal, and more preferably humans.

The phrase “other agents or therapies” is meant to describe additional medicinal compounds or treatments that are administered in treating a prostate cancer patient. Typical treatments for cancer involve chemotherapy and/or bone marrow transplantation and/or radiation therapy. Other types of therapies include radiation therapy, which involves the use of high energy rays.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use. Each carrier must be “acceptable” in the sense of being compatible with other ingredients of the formulation and not injurious to the patient.

We studied the cytotoxic effects of camalexin in prostate cancer cell lines and whether this was mediated by reactive oxygen species (ROS) generation. As models, we utilized LNCaP and its aggressive sublime, C4-2, as well as ARCaP cells stably transfected with empty vector (Neo) or constitutively active Snail cDNA that represents an epithelial mesenchymal transition (EMT) model and displays increased cell migration and tumorigenicity. We confirmed previous studies showing that C4-2 and ARCaP Snail cells express more ROS than LNCaP and ARCaP Neo, respectively. Camalexin increased ROS, decreased cell proliferation, and increased apoptosis more significantly in C4-2 and ARCaP Snail cells, as compared to LNCaP and ARCaP Neo cells, respectively, while PrEC normal prostate epithelial cells were unaffected. Increased apoptosis and mitocasp activity (which assesses caspase-3/7 activity) was associated with increased cleaved PARP apoptotic marker as shown by western blot analysis. The ROS scavenger N-acetyl cysteine (NAC) antagonized the effects of camalexin, whereas addition of exogenous hydrogen peroxide potentiated the effects of camalexin, showing that camalexin is mediating its effects through ROS.

We made the following observations regarding ROS production, cell migration, and cell viability.

ROS is higher in aggressive prostate cancer cells as compared to less aggressive prostate cancer cells and is increased further by camalexin treatment

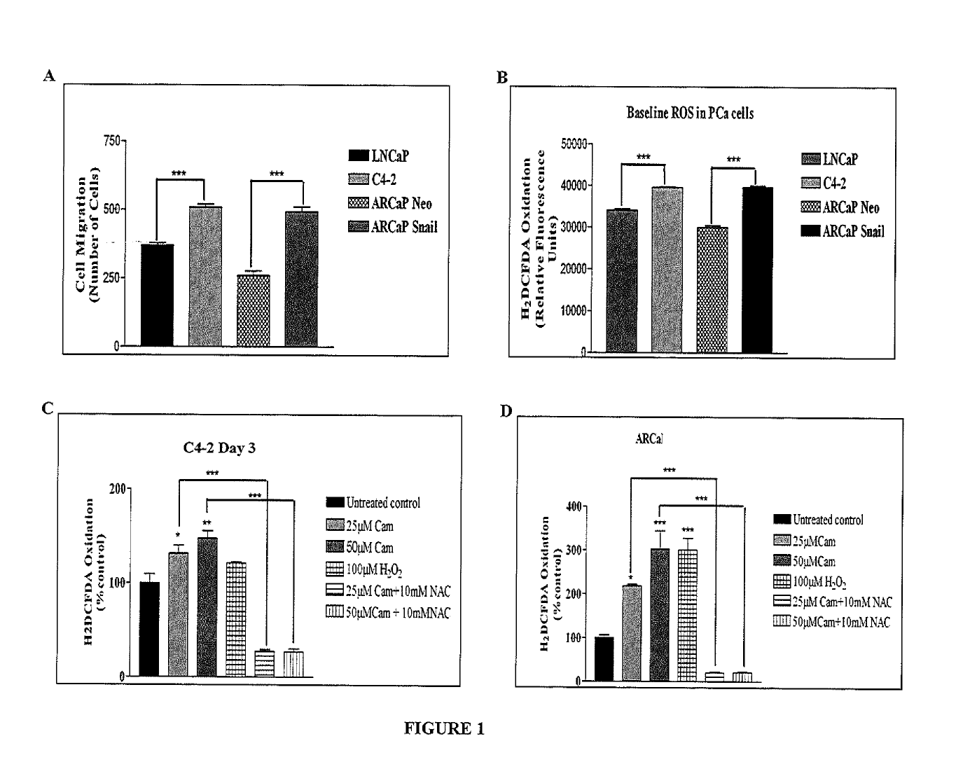

ROS has been associated with increased prostate cancer and we sought to confirm previous studies showing that more aggressive cancer cells express more ROS. ARCaP cells overexpressing Snail have been shown to increase tumorigenicity as compared to ARCaP Neo control cells and we confirmed that ARCaP Snail cells are more migratory on collagen as compared to ARCaP Neo (***p<0.001) (FIG. 1A). LNCaP-C4-2 cells portray a progression model of prostate cancer and we confirmed that C4-2 cells were more migratory than LNCaP cells (***p<0.001) (FIG. 1A).

Baseline determination of levels of ROS in untreated prostate cancer cells showed that ARCaP Snail and C4-2 (more aggressive cells) had increased endogenous ROS versus the less aggressive cells ARCaP Neo and LNCaP (***p<0.001) (FIG. 1B). We examined whether camalexin would increase ROS production in prostate cancer cells. ARCaP Snail and C4-2 cells were treated with 25 or 50 μM camalexin, and 100 μM H₂O₂ was included as a positive control. For C4-2 cells, 25 or 50 μM camalexin increased ROS by approximately 130±5% (*p<0.05) and 150±5% (**p<0.01) of untreated controls, respectively (FIG. 1C). Addition of ROS scavenger, NAC, significantly decreased camalexin-mediated ROS (FIG. 1C). As was expected, the positive control 100 μM H₂O₂ significantly increased ROS in cells (FIG. 1C), Similarly, 25 or 50 μM camalexin increased ROS by approximately 210% (p<0.05) or 350±25% (***p<0.001) in ARCaP Snail cells respectively as compared to untreated controls, which was abrogated by NAC (FIG. 1D). Therefore, ARCaP Snail and C4-2 cells are more migratory and express more ROS than ARCaP Neo and LNCaP cells, respectively, and camalexin can further increase ROS levels.

Camalexin treatments induced a more significant decrease in cell proliferation in the more aggressive prostate cancer cells as compared to the lesser aggressive cells

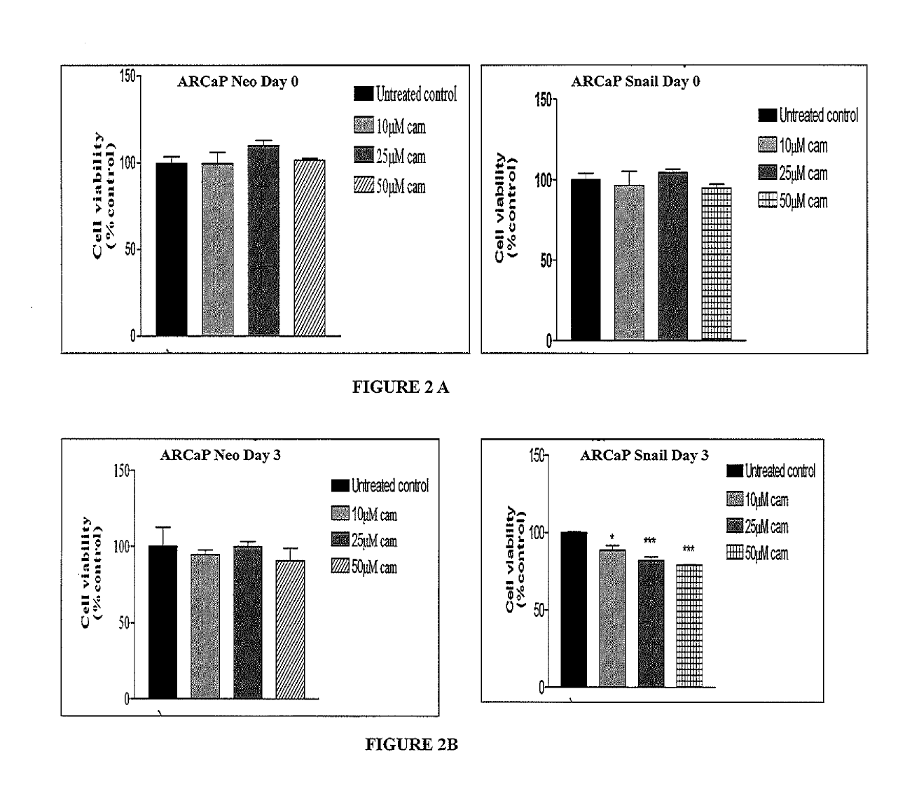

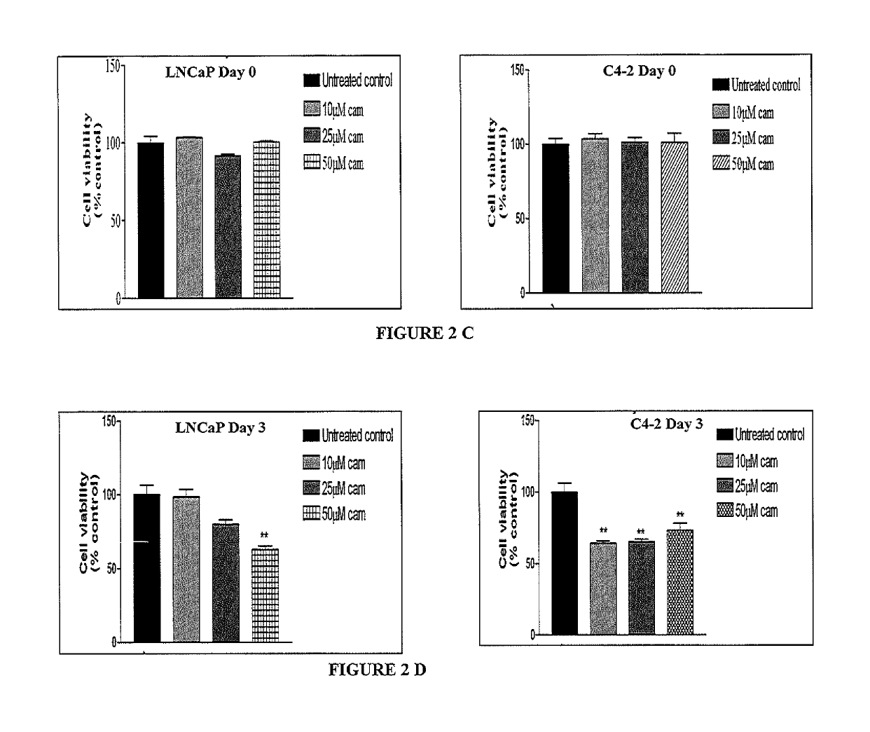

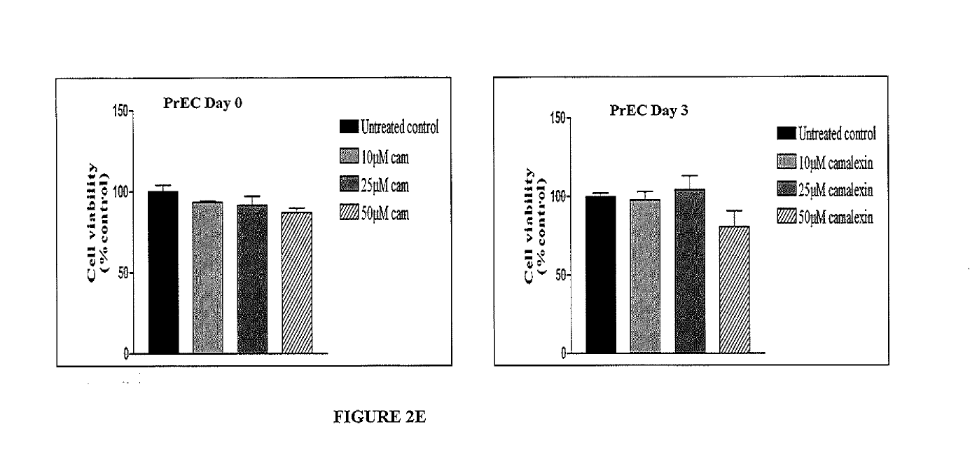

In order to test whether camalexin can affect cell viability we utilized the CellTiter 96® AQᵤₑₒᵤₛ One Solution Cell Proliferation Assay (MTS assay) on camalexin treated prostate cancer cells (ARCaP Neo, ARCaP Snail, LNCaP, C4-2) and normal prostate epithelial cells (PrEC). On day 0, both ARCaP Neo and ARCaP Snail cells showed no change in viability as expected (FIG. 2A), however, on day 3, camalexin treatment of ARCaP Snail decreased cell viability by approximately 25% (10 μM, p<0.05) and 35% (25 and 50 μM, p<0.001), while ARCaP Neo cells remained unaffected (FIG. 2B). Similarly, at day 0 for both LNCaP and C4-2 cells, viability was unaffected (FIG. 2C) but on day 3, 50 μM camalexin decreased cell viability in LNCaP by approximately 40±2% (p<0.01), while 10, 25, and 50 μM camalexin decreased C4-2 cell viability by approximately 40±5% (p<0.01) (FIG. 2D), Interestingly, normal prostate epithelial cells (PrEC) were not significantly affected by camalexin treatments at days 0 and 3 (FIG. 2E). Hence, the normal prostate epithelial cells (PrEC) and the lesser aggressive prostate cancer cells (LNCaP and ARCaP Neo) are less sensitive to camalexin treatments as compared to the more aggressive cancer cells (C4-2 and ARCaP Snail).

Camalexin treatment induces caspase expression/activity and cleaved PARP in the aggressive cell lines without altering Snail expression

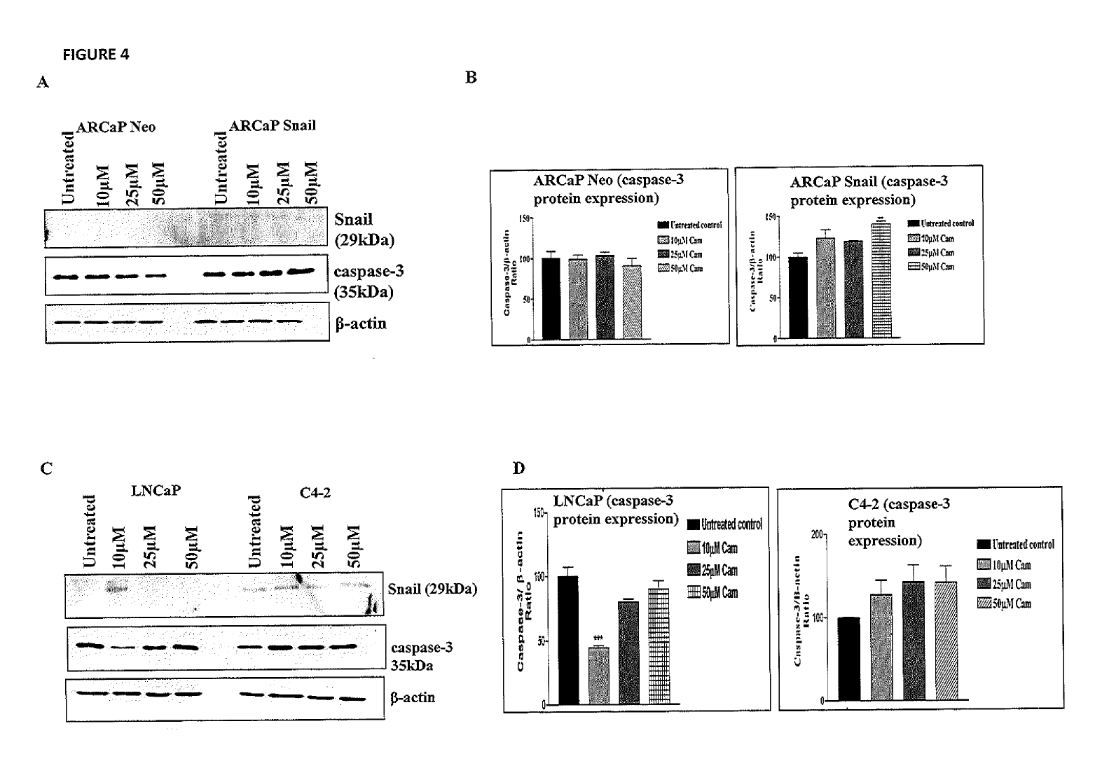

Due to camalexin's ability to significantly decrease viability especially in the more aggressive prostate cancer cells, we wanted to further demonstrate its apoptotic cytotoxic effect and also find out if camalexin could affect Snail expression. Thus, we conducted western blot analysis of lysates from untreated and 3-day camalexin treated (10, 25 and 50 μM) ARCaP Neo and LNCaP cells (less aggressive), and ARCaP Snail and C4-2 cells (more aggressive) for detection of Snail and total caspase-3 protein expression. In ARCaP Neo cells, no detectable levels of Snail protein expression were noted for untreated control and camalexin treatments while Snail was expressed in ARCaP Snail cells, and was not affected by camalexin treatments (FIG. 4A). LNCaP cells expressed only very low levels of snail protein while C4-2 cells expressed more Snail and generally camalexin treatment did not affect Snail expression except in 10 μM camalexin-treated LNCaP cells which produced a moderate expressing band for Snail protein (FIG. 4C). Interestingly, camalexin induced an increase in total caspase-3 protein levels in the more aggressive ARCaP Snail (**p<0.01 for the 50 μM treatment) and C4-2 cells but not in the less aggressive ARCaP Neo or LNCaP cells (FIGS. 4A-4D). In fact, in the lesser aggressive LNCaP cells, the 10 μM treated cells showed a significant decrease in protein expression (***p<0.001, FIG. 4D).

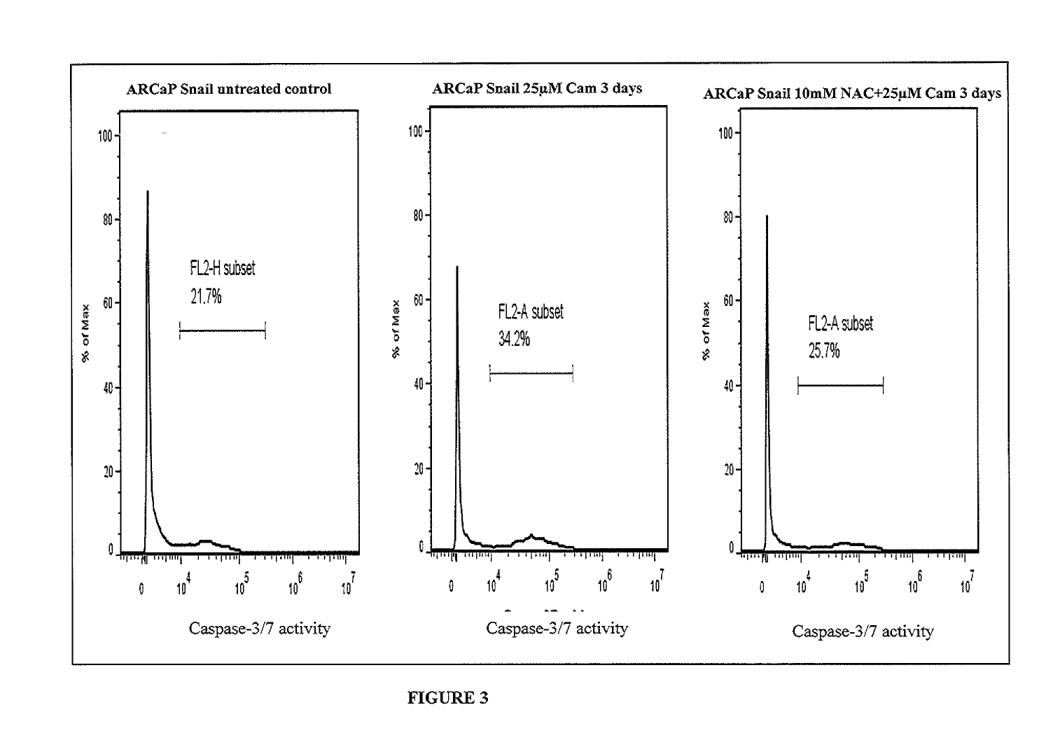

In addition to looking at total caspase levels, we next investigated caspase-3/7 activity upon camalexin treatment and whether NAC could antagonize the effects of camalexin on caspase activity. We conducted FACS analysis of caspase-3/7 activity in ARCaP Snail cells. Subsequent to 3-day camalexin treatments, a greater fraction of ARCaP Snail cells showed increased caspase-3/7 activity (34.2%) versus the untreated control cells (21.7%) (FIG. 3). Interestingly, the addition of the H₂O₂ scavenger NAC to the camalexin treated cells abrogated the percent increase in caspase-3/7 activity, almost returning it to the untreated control levels (FIG. 3). Overall, these results suggest that camalexin can increase cell apoptosis by activating caspase levels and activity in ARCaP Snail and C4-2 cells; these effects are mediated through ROS since NAC could inhibit this activity.

Increased caspase-3/7 activity induced cleavage of one of its downstream targets, PARP

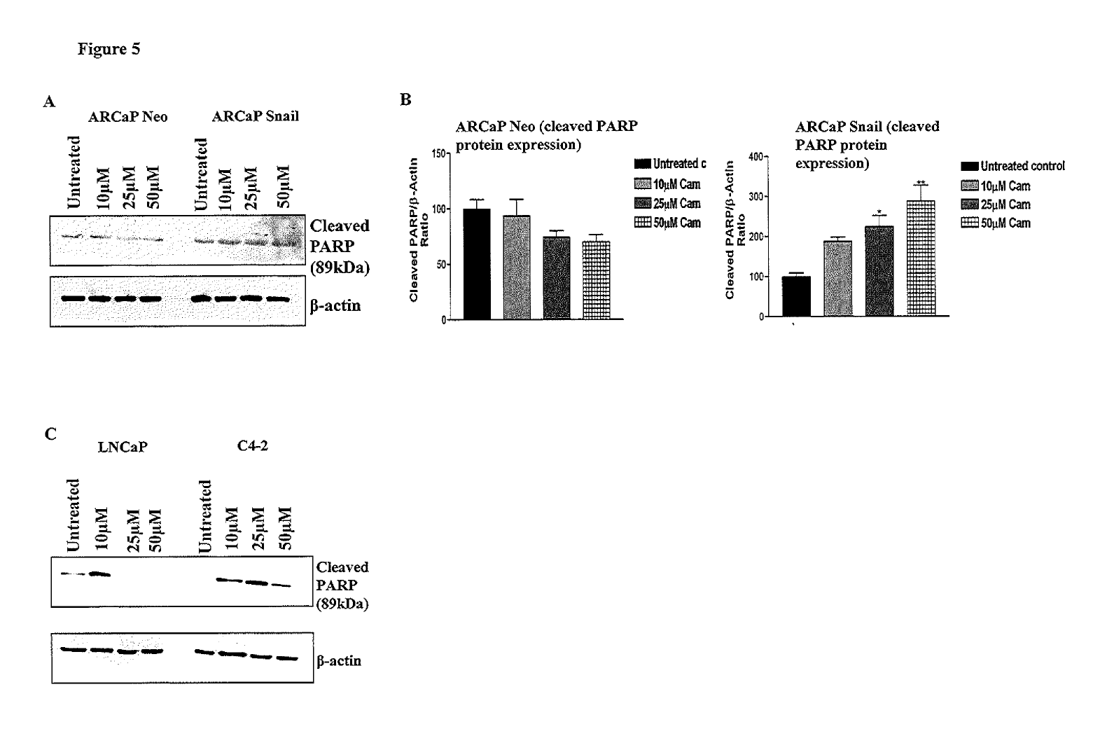

The cleavage of known caspase-3/7 substrate PARP was determined via western blot analysis of treated and untreated ARCaP Neo, ARCaP Snail, LNCaP, and C4-2 cells. Following camalexin exposure, ARCaP Snail cells (more aggressive cells), showed marked increase in levels of cleaved PARP protein expression at all treatments as compared to the untreated control with significance at the 25 and 50 μM camalexin treatments (*p<0.05, **p<0.01 respectively) (FIGS. 5A and 5B). However, ARCaP Neo cells (lesser aggressive) comparatively produced very low levels of cleaved PARP protein expression (FIGS. 5A and 5B). Likewise, the more aggressive C4-2 cells showed marked increase in PARP protein cleavage at all 3 camalexin treatments vs. the lesser aggressive LNCaP cells (FIG. 5C). Therefore, camalexin can induce PARP cleavage more significantly in ARCaP Snail and C4-2 cells than in the lesser aggressive ARCaP Neo and LNCaP cells.

NAC antagonizes camalexin mediated decrease in cell viability in aggressive prostate cancer cells

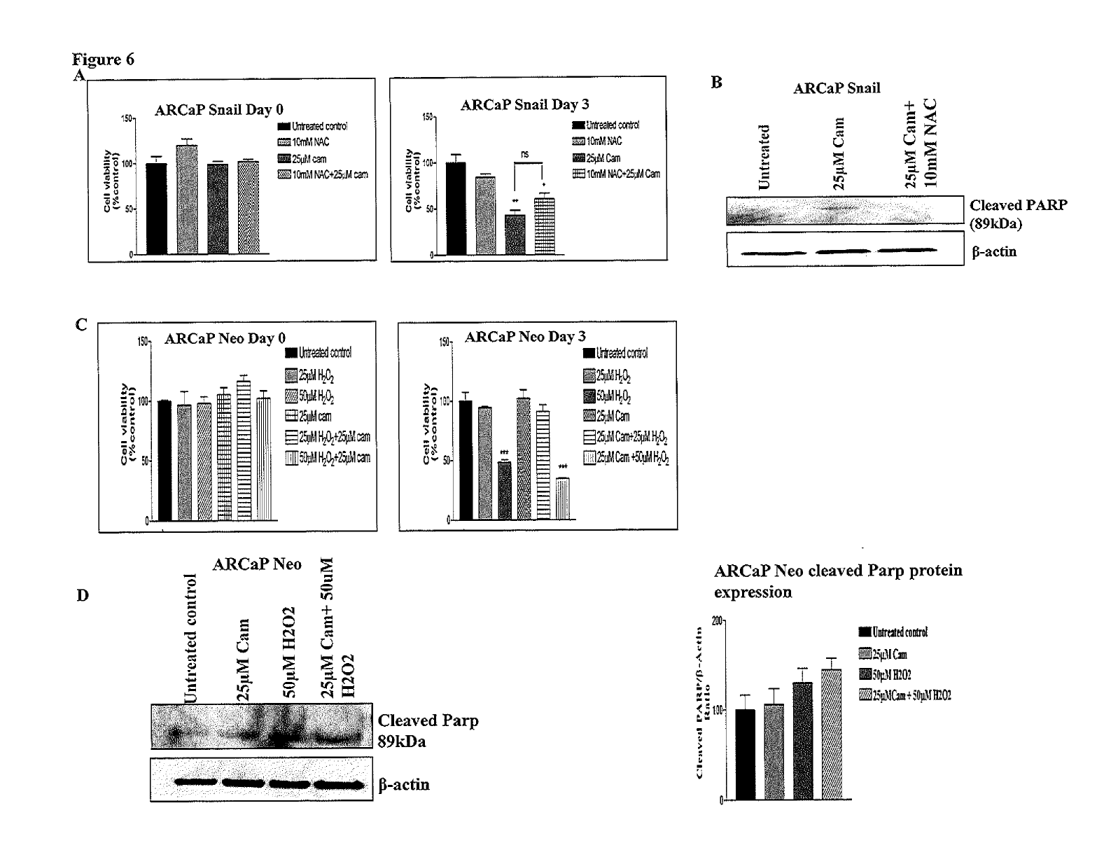

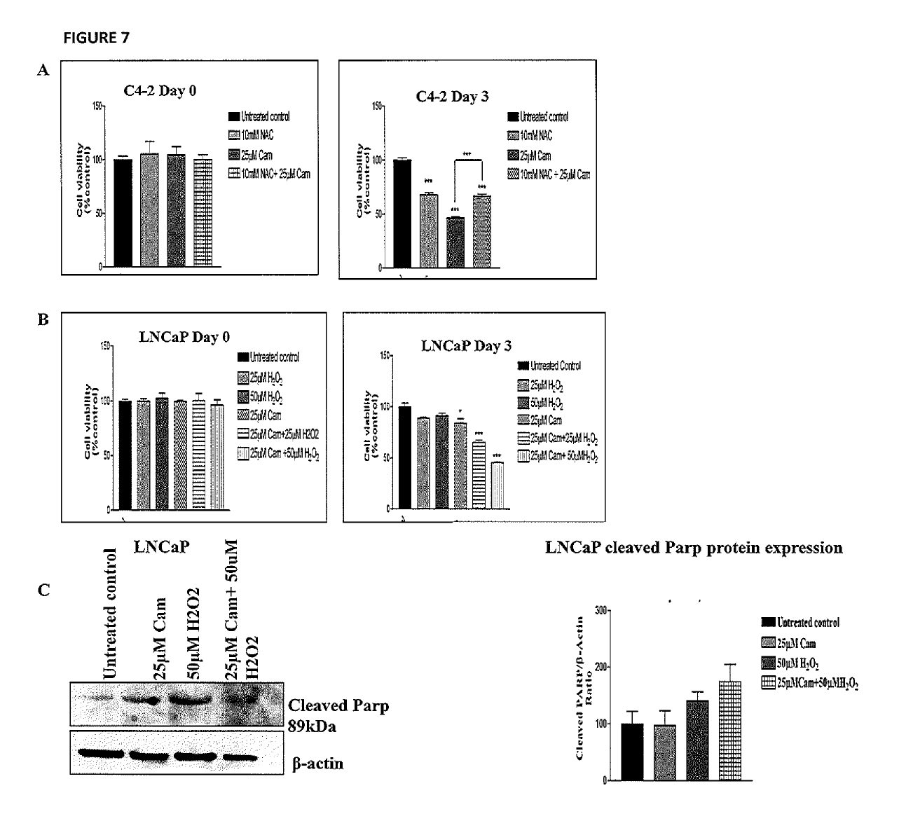

Next, we performed experiments to confirm that camalexin is mediating its effects on cell viability through ROS. Since we had observed that camalexin can decrease cell viability, we tested whether treatment with the ROS scavenger NAC would abrogate this effect and preserve viability in camalexin-treated ARCaP Snail and C4-2 cells. We observed after 3 days that camalexin-mediated decrease in cell viability was blocked by co-treatment with 10 mM NAC in ARCaP Snail which showed approximately 15% increased viability in response to NAC vs. 25 μM camalexin-only treatment (FIG. 6A). Although no statistical significance was noted for ARCaP Snail cells, the data strongly indicates a trend towards preservation of viability subsequent to addition of NAC to camalexin-treated cells. Similar results were obtained for C4-2 cells; there was approximately 25% increased viability in the 25 μM camalexin plus 10 mM NAC as compared to 25 μM camalexin-only treatment (***p<0.01) indicating that the anti-oxidant did significantly protect viability (FIG. 7A). We also evaluated cleaved PARP protein expression via western blot analysis. Our results indicated increased cleaved PARP protein expression in camalexin-treated ARCaP Snail cells could be inhibited by co-treatment with NAC (FIG. 6B). Therefore, camalexin decreases cell viability via ROS.

Addition of exogenous ROS potentiates the effect of camalexin in less aggressive prostate cancer cells

Alternatively, we examined whether adding exogenous ROS to less aggressive cells that had been relatively resistant to camalexin would now render them sensitive to camalexin. ARCaP Neo and LNCaP cells were exposed for up to 3 days to 25 μM camalexin, 25, 50 μM H₂O₂, and 50 μM H₂O₂ in combination with 25 μM camalexin. For ARCaP Neo, after 3 days exposure to treatments, only 50 μM H₂O₂ and 25 μM camalexin plus 50 μM H₂O₂ decreased viability by approximately 50 and 60% respectively (***p<0.001) (FIG. 6C) Similar results were obtained for LNCaP cells (combination treatment with H₂O₂ and camalexin was more effective at decreasing cell viability by approximately 35±1% for 25 μM camalexin plus 25 μM H₂O₂ (p<0.001) and 40% for 25 μM camalexin plus 50 μM H₂O₂ (p<0.001) (FIG. 7B). Induction of oxidative stress by treatment with H₂O₂ potentiated camalexin-mediated PARP cleavage as shown by western blot analysis in ARCaP Neo cells (FIG. 6D) and LNCaP cells (FIG. 7C). Hence, addition of H₂O₂ rendered the less aggressive cells sensitive to camalexin as shown by decreased cell viability and increased PARP cleavage.

In one aspect of the invention, camalexin is used to treat cancer. A therapeutically effective amount of camalexin is administered to the patient. Camalexin may be administered alone or in combination with other agents or therapies, preferably another cancer agent or therapy. The inventive composition may precede or follow the other agent or therapy by intervals ranging from minutes to weeks.

Pharmaceutical compositions can be prepared from camalexin in combination with other active agents, if desired, and one or more inactive ingredients such as pharmaceutically acceptable carriers as set forth below.

The pharmaceutical compositions may be employed in powder or crystalline form, in liquid solution, or in suspension. The compositions are desirably administered orally; however, they may be also administered parenterally by injection. Compositions for injection may be prepared for a desired dosage form or dose container. The injectable compositions may take such forms as suspensions, solutions or emulsions, or emulsions in oily or aqueous vehicles, and may contain various formulating agents. In injectable compositions, the carrier is typically comprised of sterile water, saline or other injectable liquid, e.g., peanut oil for intramuscular injections. Also various buffering agents, preservatives and the like can be included.

Oral formulations may take such forms as tablets, capsules, oral suspensions and oral solutions. The oral compositions may utilize carriers such as conventional formulation agents, and may include sustained release properties as well as rapid delivery forms. The dosage to be administered depends to a large extent on a variety of factors, including the condition, size and age of the subject being treated, the route and frequency of administration, and the renal and hepatic function of the subject. An ordinarily skilled physician can readily determine and prescribe the effective amount of camalexin required to treat the cancer.

Determination of a therapeutically effective amount may be readily made by the clinician, as one skilled in the art, by the use of known techniques and by observing results obtained under analogous circumstances. The dosages may be varied depending upon the requirements of the patient in the judgment of the attending clinician and the severity of the condition being treated. Suitable dosage ranges for camalexin based on body weight may range from about 10 to 100 mg per kg body weight per day (mg/kg/day).

The following examples more fully illustrate the preferred embodiments of the invention. They should in no way be construed; however, as limiting the broad scope of the invention, as described herein.

### EXAMPLES

### Reagents and Antibodies

Camalexin (40 mM in absolute ethanol) was synthesized as described in Ayer W A, et al., (1992) Synthesis of camalexin and related phytoalexins. Tetrahedron 48: 2919-2924.

Growth media RPMI 1640 (1× with L-glutamine, and without L-glutamine and phenol red), and penicillin-streptomycin were from Mediatech (Manassas, Va.). G418 was from EMD Corp Biosciences, Brookfield, Wis. Fetal bovine serum (FBS) and charcoal/dextran treated PBS (DCC-FBS) were from Hyclone, South Logan, Utah. Trypsin/EDTA was from Mediatech, Inc., Manassas, Va. MTS Cell Titer 96 Aqueous One Solution reagent was from Promega Corporation, Madison, Wis. N-acetyl cysteine (NAC) and mouse monoclonal anti-human actin antibody were from Sigma-Aldrich Inc., St Louis, Mo. Rabbit monoclonal anti-cleaved poly (ADP-ribose) polymerase (PARP) primary antibody was from Cell Signaling Technology, Inc., Danvers, Mass. Rabbit polyclonal anti-caspase3 primary antibody was from Santa Cruz Biotechnology (Santa Cruz, Calif.). HRP conjugated sheep anti-mouse, donkey anti-rabbit secondary antibodies and the enhanced chemiluminescence (ECL) western blotting detection reagent were from GE Healthcare UK Limited and Nitrocellulose membranes were from Bio-Rad (1620115, Germany). Hydrogen peroxide (H₂O₂) ACS 30% was from Acros, Fairlawn, N.J. Dichlorofluorescein (H₂DCFDA) and Alexa Fluor® 488 Annexin V/Dead Cell Apoptosis Kit were from Invitrogen, Eugene, Oreg. Dual Sensor Mitocasp® Kit was from Cell Technology, Mountain View, Calif.

### Cell Lines and Culture

Five cell lines were used: human prostate cancer cell line ARCaP stably transfected with constitutively active Snail cDNA (ARCaP Snail) representing an aggressive cell line; human prostate cancer cell line ARCaP stably transfected with empty vector Neo (ARCaP Neo) representing the lesser aggressive cell line; LNCaP cells with lesser metastatic potential; C4-2 cells—a more aggressive subline of LNCaP cells; and normal prostate epithelial cells (PrEC).

ARCaP Snail and ARCaP Neo cells were generated using the procedures outlined in Odero-Marah V A, et al., Cell Res. 2008 August; 18(8):858-70. LNCaP were obtained from ATCC and C4-2 cells were a kind gift from Dr Leland Chung, Cedar-Sinai Medical Center, Los Angeles, Calif. PrEC were obtained from ATCC, and maintained in prostate epithelial growth media (PrEBM) containing supplements and growth factors (BPE, hydrocortisone, hEGF, epinephrine, insulin, triiodothyronine, transferrin, gentamycin/amphotericin-B and retinoic acid) from Cambrex Bio Science, Walkersville, Md.

Reagents trypsin/EDTA, HEPES buffered saline solution (HEPES-BSS) and trypsin neutralizing solution (TNS) used in splitting PrEC cells were also obtained from Cambrex Bio Science, Walkersville, Md. LNCaP and C4-2 cells were routinely cultured in RPMI 1640 medium supplemented with 10% (v/v) fetal bovine serum (FBS), 2 mM L-glutamine, 50 μg/ml penicillin, and 100 μg/ml streptomycin. ARCaP Snail and ARCaP Neo cell lines had an additional 400 μg/ml G418 added to the growth media. They were grown to 70% confluence in 95% air, 5% CO₂ humidified incubator at 37° C., and routinely passaged using 0.05% trypsin/EDTA solution. For all experimental conditions RPMI 1640 without L-glutamine and phenol red and reconstituted with 5% dextran charcoal stripped FBS (DCC-FBS) was used.

### Detection of Reactive Oxygen Species

Cells were plated at a density of 1×10⁴ cells well in 96-well black clear bottom plates and left to attach overnight. Baseline ROS levels were assessed in untreated ARCaP Neo, ARCaP Snail, LNCaP, and C4-2 cells. Prior to treatment, ARCaP Snail and C4-2 cells were starved in serum-free and phenol red free RPMI. Cells were then exposed to 25 or 50 μM camalexin plus or minus 10 mM NAC, a H₂O₂ antagonist; and 100 μM H₂O₂ as a positive control. Procedures were performed following manufacturer's protocol (Molecular Probes®, Invitrogen) with slight modifications. Briefly, cells were loaded with 10 μM (final working concentration) of H₂DCFDA in pre-warmed Hanks Balance Salt solution (HBSS) for 45 minutes. This was replaced with experimental media with various treatments as well as untreated controls, for 3 days. H₂DCFDA oxidation was measured on a spectrofluorometer (BIOTek Synergy HT, BIOTeK Instruments Inc., Vt.) with excitation 485 run and emission 528 nm at desired increments.

### Migration Assay

The migratory potentials of ARCaP Neo, ARCaP Snail, LNCaP, and C4-2 cells were assayed in vitro. Cells (5×10⁴) in 0.1% BSA were plated in the upper chamber of Costar 24-well plates containing 8 μM pore size polycarbonate filter inserts coated with 3.67 μg/ul of rat tail collagen while the lower chamber contained 10% BSA. ARCaP Neo and ARCaP Snail cells were allowed to migrate for 5 hours while the less migratory LNCaP and C4-2 cells, which usually need more time, were allowed to migrate for 24 hours. Subsequently, cotton swabbing was performed to remove cells that did not migrate. Those that had migrated into the lower chamber were collected, fixed, stained with crystal violet, and counted.

### Cell Viability Assay

PrEC, ARCaP Neo, ARCaP Snail, LNCaP, and C4-2 cells were plated at a density of 2,000 cells per well in 96-well plates and allowed to attach overnight. Camalexin (10, 25 and 50 μM) was added to cells and allowed to incubate at 37° C. in a humidified 5% CO₂ atmosphere for 0 through 5 days and the cell viability was assessed daily using the CellTiter 96® Aqueous One Solution Cell Proliferation Assay according to supplier's protocol. For ROS inhibition studies, aggressive cell lines ARCaP Snail and C4-2 were treated with 10 mM NAC plus 25 μM camalexin for 3 days and cell viability was assessed daily. LNCaP and ARCaP Neo cells (less aggressive cells) were similarly exposed to 25 and 50 μM H₂O₂ in combination with camalexin to induce oxidative stress, and again, cell viability was assessed daily.

### Apoptosis Assay

Apoptosis was confirmed and quantified by measurement of externalized phosphatidylserine (PS) residues on cell membrane using the Alexa Fluor 488 Annexin/Dead cell Apoptosis kit (Molecular Probes, Invitrogen). Induction of apoptosis promotes externalization of PS (that normally is internalized), and it is thus available to bind to Annexin V-fluorescent dye conjugate. Propidium iodide (PI) was used to distinguish viable and apoptotic cells from necrotic cells since viable and apoptotic cells will exclude PI while membrane-damaged necrotic cells are permeable to PI. Cells that stained positive for Annexin V-Alexa Fluor 488 and negative for PI (Alexa Fluor 488⁺/PI⁻) were considered to be undergoing early apoptosis; cells that stained positive for both Alexa Fluor 488 and PI were considered as either in late apoptosis or necrosis. Cells that stained negative for both Alexa Fluor 488 and PI were considered non-apoptotic (viable). ARCaP Neo, ARCaP Snail, LNCaP, and C4-2 cells were treated with 25 μM camalexin for 3 days and the apoptosis assay was performed using Accuri® C6 Flow Cytometer (Accuri Cytometers Inc., Ann Arbor, Mich.) following the manufacturer's protocol. Experiments were performed 3 times and a representative result displayed in the form of PI vs. Annexin V-Alexa Fluor 488 fluorescence scattergrams.

### Western Blot Analysis

Western blot was performed as described in Odero-Marah V A, et al., (2008) Cell Res 18(8): 858-870. Briefly, cells were plated at a density of 2×10⁶ cells in 75 cm² flasks, and allowed to attach overnight. Prior to treatments, ARCaP Neo and ARCaP Snail cells were serum-starved in serum-free and phenol red free RPMI medium overnight, while LNCaP and C4-2 cells were serum-starved for 4 hours. Cells were then lysed with buffer containing 1×RIPA, with protease inhibitors (Aprotinin 0.1 mg/ml, PMSF 1 mM, Leupeptin 0.1 mM, pepstatin A 0.1 mM) and phosphatase inhibitor (10 mM Sodium Orthovanadate) and centrifuged at 13,000 rpm. Total protein content was determined using the BCA Protein Assay Kit (Thermo Scientific, Rockford, Ill.) following protocol as per manufacturer's specifications. Cell lysates (40-50 μg) were subjected to SDS-Polyacrylamide gel electrophoresis (SDS-PAGE) and then transferred to pure nitrocellulose membrane (Bio-Rad Laboratories). Western blot analysis for Snail, caspase 3, and cleaved PARP protein expression was then performed. Anti-mouse and anti-rabbit IgG Horseradish Peroxidase in blocking solution (5% nonfat milk in TBS-TB) was employed as secondary antibodies for chemiluminescent detection. Blots were visualized with chemiluminescence ECL detection system (Pierce, Rockford, Ill.) and analyzed using FUJIFILM LAS 3000 imager and dark room x-ray film to observe protein expression. To evaluate protein loading, membranes were immediately stripped with Restore™ Western Blot Stripping Buffer (Thermo scientific, USA) and reprobed for β-actin as a loading control.

### Measurement of Caspase-3/7 Activation

Caspases are generally accepted as the executioners of apoptosis. We treated ARCaP Snail cells with 25 μM camalexin alone and in combination with 10 mM NAC, for 3 days and performed FACS analysis of caspase 3/7 activity in ARCaP Snail cells using the Dual Sensor: Mitocasp™ assay (MitoCap200-1 kit, Cell Technology Inc., Mountain View Calif., USA) according to the manufacturer's protocol. Stained cells were evaluated in an Accuri® C6 Flow Cytometer (Accuri Cytometers Inc., Ann Arbor, Mich.) and analyzed using FlowJo software.

### Statistical Analysis

Statistical Analysis was done using ANOVA and Tukey's Multiple Comparison as Post Hoc Test from Graph Pad Prizm3 software (*p<0.05, **p<0.01, ***p<0.001). Values were normalized to untreated controls and expressed as mean±S.E.M (N=3).

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A method of treating prostate cancer comprising administering a therapeutically effective dose of camalexin.

2. The method of claim 1 wherein the camalexin is administered in combination with one or more additional anti-cancer active agents or therapies.

3. The method of claim 1 wherein the camalexin is administered in a pharmaceutical composition comprising a pharmaceutically acceptable carrier.

4. The method of claim 1 wherein camalexin increases ROS production by prostate cancer cells.

5. The method of claim 1 wherein camalexin increases ROS production in aggressive cancer cells to a level to trigger apoptosis.

6. The method of claim 1 wherein camalexin works through ROS production.

7. The method of claim 1 further comprising the addition of exogenous ROS.

8. The method of claim 1 wherein camalexin decreases prostate cancer cell viability.

9. The method of claim 8 where the effect of camalexin on aggressive prostate cancer cells is greater than on les aggressive prostate cancer cells.

10. A method of decreasing the viability of prostate cancer cells comprising contacting the cells with an effective amount of camalexin.

11. The method of claim 10 wherein camalexin increases ROS production of the prostate cancer cells.

12. The method of claim 10 wherein the viability of aggressive prostate cancer cells is decreased to a greater extent than the viability of less aggressive prostate cancer cells.

13. The method of claim 10 further comprising the addition of exogenous ROS.

14. A method of increasing ROS production in prostate cancer cells by exposing them to an effective amount of camalexin.

15. The method of claim 14 wherein the increase in ROS causes a decrease in cell viability.
